(19) United States
(12) Patent Application Publication
(10) Pub. No.: US 2013/0058954 A1
(43) Pub. Date: Mar. 7, 2013

(54) DIAGNOSTIC AND THERAPEUTIC METHODS FOR CORNEAL ECTASIA FOLLOWING REFRACTIVE SURGERY, KERATOCONUS OR PELLUCID DEGENERATION

(71) Applicants: Gerard Sutton, Balmoral (AU); John McAvoy, Stanmore (AU); Michele Madigan, Hurlstone Park (AU)

(72) Inventors: Gerard Sutton, Balmoral (AU); John McAvoy, Stanmore (AU); Michele Madigan, Hurlstone Park (AU)

(73) Assignee: THE UNIVERSITY OF SYDNEY, Sydney, NSW (AU)

(21) Appl. No.: 13/513,800

(22) PCT Filed: Dec. 2, 2010
(86) PCT No.: PCT/AU10/01633

§ 371 Date: Nov. 21, 2012

(30) Foreign Application Priority Data
Dec. 2, 2009 (AU) 2009905883

(51) Int. Cl. C40B 30/00; A61K 31/7052; A61P 27/02; A61K 33/00; A61K 31/5377; A61K 39/395; A61K 31/437

(52) U.S. Cl. 424/172.1; 506/7; 514/44 A; 514/303; 424/722; 514/234.2

(57) ABSTRACT

The present invention relates to methods of diagnosis and treatment of corneal ectasia following refractive surgery, keratoconus or pellucid marginal degeneration in a subject by determining or modulating the level of expression of molecules associated with the Wnt signalling pathway. Marker molecules of the present invention include SFRP1, PITX2, LEF1, WNT16 and WNT5A.

### RELATED APPLICATION

This application claims benefit from Australian provisional patent application number 2009905883, filed 2 Dec. 2009, entitled “Diagnostic and Therapeutic Methods”, the entire disclosure of which is incorporated herein by reference.

### TECHNICAL FIELD

The present invention relates to methods of diagnosis and treatment of corneal ectasia following refractive surgery or keratoconus or pellucid marginal degeneration in a subject.

### BACKGROUND OF THE INVENTION

Keratoconus is a bilateral progressive, non-inflammatory but degenerative ectasia of the cornea which usually presents in the second decade of life and progresses into the third and fourth decade with a decreasing visual function. It is rare for progressive thinning and protrusion of the cornea to continue beyond that period. Keratoconus causes loss of visual function due to corneal thinning, irregular astigmatism and progressive myopia. The alteration to corneal topography occurs very early in the disease.

Keratoconus is normally diagnosed when vision deteriorates and the patient seeks an opinion from optometrist or ophthalmologist. Keratometry or corneal imaging may be used to diagnose keratoconus in the early stages. Classical clinical features such as Vogt's striae or the presence of a Fleischer ring on slit lamp examination are also pathognomonic of the disease. Diagnosis may be difficult if the disease affects one eye only which is common in the early stages, and accordingly in some subjects the time of diagnosis can occur relatively late in the progression of the disease.

The successful diagnosis of keratoconus is important in the screening of subjects intending to undergo refractive surgery. Subjects with early signs of keratoconus should be excluded from common refractive surgery procedures, as the corneal reshaping that occurs in these procedures may develop into subsequent progressive corneal ectasia. There is thus a need to identify keratoconus in subjects who might not yet exhibit all of the common clinical manifestations of keratoconus.

The rate of progression of keratoconus varies between subjects, with some subjects having a relatively rapid degeneration of vision such that therapeutic intervention may be necessary. If disease progression can be identified, riboflavin-induced collagen cross-linking treatment may in some cases be used to slow or halt the progression of corneal thinning. In other subjects, the progression of keratoconus is relatively slow and the corneal shape may largely stabilize without therapeutic intervention.

The aetiology of keratoconus is unclear but recent studies indicate possible roles for oxidative damage and keratocyte apoptosis.

In the light of the development of therapeutic interventions for keratoconus, there is a need for a sensitive method for the diagnosis of keratoconus, either prior to the onset of gross clinical symptoms such as corneal topography changes or after the onset of clinical symptoms. In addition, there is a need for alternative methods for treating or preventing the progression of keratoconus in a subject.

### SUMMARY OF THE INVENTION

The Wnt signalling pathway describes a complex network of proteins involved in a cascade that controls many physiological processes in mammals, including a role in apoptosis. The canonical Wnt signalling pathway involves Wnt proteins binding to cell-surface receptors of the Frizzled family, causing receptors to activate Dishevelled proteins, eventually regulating β-catenin movement to the nucleus and subsequent gene expression. Since apoptosis is one pathological feature of keratoconus, the present inventors examined whether one or more defective elements in the Wnt pathway in the corneal stroma or epithelium may be involved in pathological processes of this condition.

Accordingly, in a first aspect there is provided a method of diagnosis of corneal ectasia following refractive surgery, or keratoconus, or pellucid marginal degeneration in a subject, the method comprising comparing the level of expression of a marker molecule associated with the Wnt signalling pathway by corneal cells with a control level of expression of the molecule, wherein an elevated or lower level of expression of the molecule associated with the Wnt signalling pathway in the subject indicates the subject has or is at risk of developing corneal ectasia following refractive surgery, or keratoconus, or pellucid marginal degeneration.

In certain embodiments, the corneal cells are corneal epithelial cells, conjunctival epithelial cells or keratocytes. In certain embodiments, the method comprises comparing the level of expression in a biological sample of a marker molecule associated with the Wnt signalling pathway by corneal cells with a control level of expression of the marker molecule.

In certain embodiments the elevated level of expression of the marker molecule is at least a 2 fold, or at least 3 fold, or at least a 4 fold, or at least a 5 fold or at least a 9 fold or at least a 10 fold increase in level of expression over the control level. In certain embodiments the lower level of expression is at least a 2 fold, or at least 3 fold, or at least a 4 fold, or at least a 5 fold or at least a 9 fold or at least a 10 fold lower level of expression than the control level.

Also provided is a marker molecule associated with the Wnt signalling pathway for use in the diagnosis of corneal ectasia following refractive surgery, or keratoconus, or pellucid marginal degeneration.

Also provided is use of a marker molecule associated with the Wnt signalling pathway for the preparation of a diagnostic agent for the diagnosis of corneal ectasia following refractive surgery, or keratoconus, or pellucid marginal degeneration in a subject

In certain embodiments an elevated level of expression of the molecule associated with the Wnt signalling pathway in the subject indicates the subject has or is at risk of developing corneal ectasia following refractive surgery, or keratoconus, or pellucid marginal degeneration. In this embodiment the marker molecule associated with the Wnt signalling pathway may, for example, be selected from Secreted Frizzled-related Protein 1, Paired-like homeodomain transcription factor 2, or Lymphoid enhancer-binding factor 1. In certain embodiments the marker molecule is mRNA encoding the Secreted Frizzled-related Protein 1, Paired-like homeodomain transcription factor 2 or Lymphoid enhancer-binding factor 1 polypeptide. In certain embodiments the marker molecule is Secreted Frizzled-related Protein 1, Paired-like homeodomain transcription factor 2 or Lymphoid enhancer-binding factor 1 polypeptide.

In certain embodiments a lower level of expression of the molecule associated with the Wnt signalling pathway in the subject indicates the subject has or is at risk of developing corneal ectasia following refractive surgery, or keratoconus, or pellucid marginal degeneration. In this embodiment the marker molecule associated with the Wnt signalling pathway may, for example, be selected from Wingless-type MMTV integration site family, member 16 or Wingless-type MMTV integration site family, member 5A.

In another aspect there is provided a method of treating corneal ectasia following refractive surgery, or keratoconus, or pellucid marginal degeneration in a subject, comprising administering to the subject a modulator of the Wnt signalling pathway.

Also provided is a modulator of the Wnt signalling pathway for use in the treatment of corneal ectasia following refractive surgery, or keratoconus, or pellucid marginal degeneration.

Also provided is the use of a modulator of the Wnt signalling pathway in the preparation of a medicament for use in the treatment of corneal ectasia following refractive surgery, or keratoconus, or pellucid marginal degeneration in a subject

In certain embodiments, the modulator of the Wnt signalling pathway is an agonist of the Wnt signalling pathway, such as an antagonist of Secreted Frizzled-related Protein 1, Paired-like homeodomain transcription factor 2 or Lymphoid enhancer-binding factor 1. The antagonist of Secreted Frizzled-related Protein 1, Paired-like homeodomain transcription factor 2 or Lymphoid enhancer-binding factor 1 may be an antibody, an antisense molecule or an RNAi.

In certain embodiments, the agonist of the Wnt signalling pathway is a pyrazolo[3,4-C]pyridine, such as N-(5-phenyl-1H-pyrazolo[3,4-C]pyridazino-3 yl)-4morpholine butanamide, or lithium.

### BRIEF DESCRIPTION OF THE FIGURES

Certain embodiments will now be described, by way of an example only, with reference to the accompanying figures, wherein:

FIG. 1 is a graph which demonstrates the fold up-regulation of genes associated with the Wnt signalling pathway, as measured by, RT-PCR, in the corneal epithelium of keratoconus sufferers (represented by the average of 4 subjects) compared with control corneal epithelium (represented by the average of 6 control subjects). PITX2 and SFRP1 (labelled on the graph) and LEF1 (not labelled) exhibited more than a 2 fold up regulation in keratoconus subjects. Of these markers, SFRP-1 was more dramatically up regulated.

FIG. 2A provides example fluorescent micrographs of sections of anterior cornea, including corneal epithelium, from a keratoconus subject immunolabelled with anti-SFRP-1 antibody. Cell nuclei in these sections were labelled with propidium iodide. The basal epithelial cells of the keratoconus subject exhibited SFRP1 immunolabelling, whilst the control cornea (not shown) appeared to remain unlabelled.

FIG. 2B provides example fluorescent micrographs of sections of anterior cornea, including corneal epithelium, from a control subject and a keratoconus subject immunolabelled with anti-SFRP-1 antibody. The basal epithelial cells of the keratoconus subject exhibited SFRP1 immunolabelling, whilst the control cornea appeared to remain unlabelled.

### ABBREVIATIONS

BSCVA best spectacle corrected visual acuity

KC keratoconus

LASIK laser-assisted in situ keratomileusis

LEF1 lymphoid enhancer-binding factor-1

PITX2 paired-like homeodomain transcription factor-2

PRK photorefractive keratectomy

RT-PCR Reverse transcription real-time polymerase chain reaction

SFRP1 secreted frizzled related protein-1 mRNA

SFRP1 secreted frizzled related protein-1 polypeptide

Wnt Wingless-type MMTV integration site family

### DETAILED DESCRIPTION OF THE PREFERRED EMBODIMENTS

In a first aspect there is provided a method of diagnosis of corneal ectasia following refractive surgery, or keratoconus, or pellucid marginal degeneration in a subject, the method comprising comparing the level of expression of a marker molecule associated with the Wnt signalling pathway by corneal cells with a control level of expression of the molecule, wherein an elevated or lower level of expression of the molecule associated with the Wnt signalling pathway in the subject indicates the subject has or is at risk of developing corneal ectasia following refractive surgery, or keratoconus, or pellucid marginal degeneration.

Keratoconus, Pellucid Marginal Degeneration and Corneal Ectasia

In certain embodiments, there is provided a method of diagnosis of keratoconus in a subject. Keratoconus involves progressive corneal thinning (ectasia), irregular astigmatism and progressive myopia. Corneal topographic features which are associated with established keratoconus include irregular astigmatism, usually with inferior corneal steepening, high keratometry, and corneal thinning. The “posterior float” (associated with differences in posterior and anterior corneal curvature) is often high.

Features of established keratoconus which may be observed on clinical examination of a subject include central or paracentral corneal thinning, for example when measured by corneal pachymetry, the presence of an iron deposition ring or “Fleisher ring” which is found around the edge of the cone, and the presence of vertical folds in the cornea called Vogt's Striae. The identification of corneal thinning and breaks in Bowman's membrane are considered pathognomonic of keratoconus. Breaks in Descemet's membrane may also be observed, particularly in later stages of keratoconus.

The histopathology of keratoconus is well described, with changes occurring at all levels of the cornea. Typically histopathological features exhibited will include corneal central or paracentral thinning, breaks in Bowman's membrane and in later stages Descemet's membrane and keratocyte cell death. The corneal epithelium may be thinned and when breaks in Bowman's membrane occur, the corneal epithelium may appear to grow into the underlying corneal stroma. The density of keratocytes has been shown to be reduced in keratoconic subjects when compared to controls.

The term keratoconus is also intended herein to encompass forme fruste keratoconus. It has been estimated that up to 10% of all subjects who seek refractive surgery for vision correction have forme fruste keratoconus, that is corneal topographical abnormalities without other clinical signs of keratoconus. Subjects with forme fruste keratoconus prior to refractive surgery who undergo refractive surgery techniques are at a greater risk of developing progressive corneal ectasia than subjects who do not exhibit forme fruste keratoconus.

In certain embodiments, there is provided a method of diagnosis of pellucid marginal degeneration in a subject. Corneal ectasia is observed in Pellucid Marginal Degeneration, a condition involving non-inflammatory inferior peripheral corneal thinning which produces changes to corneal shape which may be confused with keratoconus. Pellucid marginal degeneration is characterised by large degrees of central against the rule astigmatism and with the rule astigmatism in the area of thinning.

In certain embodiments, there is provided a method of diagnosis of progressive corneal ectasia following refractive surgery in a subject. Progressive corneal ectasia following refractive surgery, also described as iatrogenic keratoconus, iatrogenic keratectasia or secondary keratoconus, is a progressive bulging of the cornea accompanied by corneal thinning which may occur following refractive surgical procedures, such as photorefractive keratectomy (PRK) or more commonly in laser-assisted in situ keratomileusis (LASIK). This condition arises following LASIK because LASIK penetrates the cornea much more deeply than other surgical procedures and therefore can result in excessive thinning and structural compromise of the cornea. Factors which are known to contribute to ectasia risk include carrying out refractive surgery on a subject whose corneal thickness is not sufficient to accept the surgery, the cutting of a deeper flap in LASIK procedures than intended, and where the refractive surgery subject was suffering from forme fruste keratoconus which was not detected prior to surgery. There are both surgical and non surgical treatments to mitigate the symptoms of ectasia and attempt to prevent its progression. Gas permeable contact lenses may stabilise it in some patients. Intracorneal rings or INTACS are increasingly being used to treat ectasia; sometimes only one of the ring segments is used. Many cases of ectasia require a corneal transplant (lamellar or penetrating keratoplasty).

It is anticipated that the corneal thinning and ectasia exhibited in each of these conditions has a similar underlying mechanism and that therefore the diagnostic methods described herein will be equally applicable to all of these conditions.

Corneal ectasia following refractive surgery, keratoconus and pellucid marginal degeneration are progressive diseases, with the rate of progression varying between different subjects. In subjects with a rapid progression the ultimate visual outcome is usually worse that in subjects with a relatively slow progression, with an increase in likelihood for subjects with a rapid progression that corneal transplant will be necessary.

The rate of progression of Corneal ectasia following refractive surgery, keratoconus, or pellucid marginal degeneration is typically assessed by measuring changes in corneal steepening over a time period, such as 3 months or 6 months, using techniques such as scanning slit videokeratography or placido disk videokeratography. Corneal thinning associated with keratoconus, pellucid marginal degeneration or corneal ectasia following refractive surgery may be conveniently measured may also be assessed using scanning slit videokeratography. As described herein, a subject with rapidly progressing keratoconus or pellucid marginal degeneration or corneal ectasia following refractive surgery exhibits an increase in measured corneal steepness by at least 2 mm over a 3 month period.

Diagnostic Methods

It will be understood that the term “diagnosis” in the context of the diagnosis of corneal ectasia following refractive surgery, keratoconus, or pellucid marginal degeneration may encompass the identification that a subject has an active or established form of any of the above-mentioned conditions. In certain embodiments “diagnosis” is intended to encompass the identification that a subject is at risk of developing clinical symptoms of any of the above mentioned conditions. In certain embodiments, “diagnosis” is intended to encompass the monitoring of the degree of progression and/or the rate of progression of any of the above-mentioned conditions in a subject known to have any of the above-mentioned conditions or to be at risk of developing clinical symptoms of the above-mentioned conditions.

Subjects

The subject may be a human subject who is suspected of suffering from corneal ectasia following refractive surgery, keratoconus, or pellucid marginal degeneration, for example a subject who exhibits some of the clinical signs of keratoconus but who cannot be definitively diagnosed on the basis of these clinical signs alone. The subject may be a human subject at risk of developing of any of the above-mentioned conditions, for example a sibling of a known keratoconus sufferer and who therefore has a greater statistical risk of developing keratoconus than an aged matched subject from the general population.

As demonstrated in Example 1 described herein, the rate of progression of keratoconus in a subject appears to correlate with the level of expression of certain markers associated with Wnt signalling pathway in corneal cells taken from the subject. Accordingly, in certain embodiments the subject may be a human subject who is known to have keratoconus, in which case the diagnostic test may allow the rate of progression of keratoconus, pellucid marginal degeneration or corneal ectasia following refractive surgery to be assessed, either in the absence of treatment or during a therapeutic regimen intended to slow or reverse the onset or progression of the above-mentioned conditions.

The subject may be a deceased corneal tissue donor, in which case the purpose of diagnosis is to ensure that the recipient of the corneal tissue does not subsequently develop clinical signs of keratoconus. The subject may be intending to undergo corneal refractive surgery, such as LASIK or PRK which are contraindicated if the subject is found to have keratoconus, and who are therefore at risk of developing corneal ectasia following refractive surgery.

Samples comprising Marker Molecules

The diagnostic methods described herein rely on the comparing the level of expression of one or more marker molecules which are involved with or which are components of the Wnt signalling pathway in the cornea a subject with control levels of expression.

In certain embodiments the level of expression of the marker molecules may be measured in a subject in situ, for example by administering to the tear film of a subject a fluorescein-labelled antibody probe for the molecule of interest and examining the distribution of labelling in the corneal epithelium and/or the tears, over time if necessary, using a slit lamp and cobalt blue illumination.

In certain embodiments the level of expression of the marker molecules may be measured in vitro in an isolated cell sample from a subject. The cell sample may comprise any one or more of cells of the corneal epithelium, such as any one or more of corneal epithelial basal cells, wing cells or squamous cells, cells of the conjunctival epithelium, or corneal or conjunctival stromal cells, such as keratocytes.

In certain embodiments the cells are cells which originate from a region of corneal thinning.

Corneal epithelial cells may be obtained from a subject by mechanical debridement using a sterile corneal scraper, by impression cytology or by harvesting cells which are suspended in tear samples, such as corneal epithelial cells released into the tear film by rubbing the eyes and collected with micropipettes using standard techniques, or by any other techniques known in the art, provided that the sampling substantially preserves the detectable molecules associated with the Wnt signalling pathway, such as Secreted Frizzled-related Protein 1 (SFRP1), and/or paired-like homeodomain transcription factor 2 (PITX2) and/or lymphoid enhancer-binding factor 1 (LEF1).

In certain embodiments the cells comprise basal corneal epithelial cells and the cells are harvested from the surface of the eye by corneal scraping. In certain embodiments, the corneal epithelial cells comprise wing cells or squamous cells of the corneal epithelium.

Where the cells are stromal cells, small biopsy samples may be obtained from the cornea or conjunctiva using known sampling techniques, such as tissue sampled using a micro trephine.

In certain embodiments the marker molecule is present in a biological sample from a subject, such as a tear sample, and is not necessarily directly associated with corneal cells in the sample. For example, marker molecule polypeptides may be released or secreted into the tear film by corneal cells and may be measured in a cell-free sample, such as a tear sample containing negligable numbers of corneal cells. Tear samples may be collected using capillary tube tear sampling methods which are generally available to practitioners.

Expression of Marker Molecules

The diagnostic method involves comparing the level of expression a marker molecule in a subject with control levels of expression of these marker molecules. The comparison may involve identifying the level of expression of the marker molecule in the subject and comparing this measured level to a known control level. It will be understood that a comparison of the levels of expression of two or more marker molecules with control levels of expression is also intended to be encompassed.

The control level of expression may be determined by measuring the level of expression of the marker molecule using the same technique in a plurality of control subjects who do not exhibit clinical symptoms of keratoconus, pellucid marginal degeneration or corneal ectasia following refractive surgery. In particular embodiments the plurality of control subjects are a plurality of age-matched control subjects.

The control level of expression may be determined by measuring the level of expression of the molecule in a population of subjects with identified and clinically assessed corneal ectasia following refractive surgery, keratoconus or pellucid marginal degeneration in order to generate a standard curve of expression level compared to clinical severity and/or the rate of clinical progression.

The comparison may be a comparison of whether the level of expression of the one or more marker molecules of the subject is greater than the control level of expression. In certain embodiments the comparison is a comparison of whether the level of expression of the one or more marker molecule of the subject is at least 2-fold greater than the control level, or at least 3-fold greater, or at least 4-fold greater, or at least 5-fold greater, or at least 10-fold greater or at least 20-fold greater than the control level. When making such a comparison, the control level of expression of the marker molecule is assigned an arbitrary level of 1. If, for example, the marker molecule from a test subject sample is present in twice the amount than present in a control sample it would be considered to be elevated 2 fold in the test subject. On the other hand, if for example, the marker molecule from a test subject sample is present in half the amount of a control sample it would be considered to be expressed 2 fold less than the control.

The comparison may be a comparison of whether the level of expression of the marker molecule of the subject is less than the control level. The comparison may be a comparison of whether the level of expression of the certain molecule of the subject is at least 2-fold less than the control level, or at least 3-fold less, or at least 4-fold less, or at least 5-fold less, or at least 10-fold less than the control level.

In certain embodiments the comparison of the level of expression of a marker molecule involves a comparison of the level of expression of a polypeptide marker molecule. In certain embodiments the comparison of the level of expression of a marker molecule involves a comparison of the level of expression of a polynucleotide marker molecule.

When determining the level of expression of a polypeptide or polynucleotide marker molecule, in some circumstances it may be advantageous to normalise the detected amount of expressed polypeptide or polynucleotide marker against the detected level of expression of one or more “housekeeping” genes from the same subject which are expressed in substantially invariant amounts, such as beta-2-macroglobulin, hypoxanthine phosphoribosyltransferase, ribosomal protein L13a, glyceraldehyde-3-phosphate dehydrogenase or beta actin genes.

The comparison may involve a statistical comparison between the normalised level of expression of marker molecules in a subject with a control level. In certain embodiments the statistical comparison is a T-test, and in a marker which is upregulated or down regulated in keratoconus, pellucid marginal degeneration or corneal ectasia following refractive surgery the p-value of the statistical comparison is <0.05.

Marker Molecules

In embodiments related to the diagnosis of keratoconus or pellucid marginal degeneration or corneal ectasia following refractive surgery, the marker molecules are involved with or are components of the Wnt signalling pathway. In certain embodiments the marker molecule which is diagnostic for keratoconus or pellucid marginal degeneration or corneal ectasia and which is associated with the Wnt signalling pathway is a component of the signalling pathway. In other embodiments the marker molecule may be involved with but may not play a direct role in the signalling pathway, but may for example agonise or antagonise one or more components of the signalling pathway. An example of such an embodiment is the Wnt signalling pathway antagonist Secreted Frizzled-related Protein 1 (SFRP1).

Secreted Frizzled-related Protein-1 or SFRP1 as described herein may be a polynucleotide, such as a cDNA sequence or an mRNA sequence of SFRP1 (abbreviated throughout the specification as SFRP1), or may be the SFRP-1 polypeptide. Accordingly the “expression” of Secreted Frizzled-related Protein-1 may refer to the expression of Secreted Frizzled-related Protein-1 cDNA, mRNA and/or Secreted Frizzled-related Protein-1 polypeptide.

Similarly, paired-like homeodomain transcription factor 2 (PITX2) as described herein may be a polynucleotide, such as a cDNA sequence or an mRNA sequence of PITX2 (abbreviated throughout the specification as PITX2), or may be the PITX2 polypeptide, and lymphoid enhancer-binding factor 1 or LEF1 as described herein may be a polynucleotide, such as a cDNA sequence or an mRNA sequence of LEF1 (abbreviated throughout the specification as LEF1), or may be the LEF1 polypeptide.

Similarly, marker molecules Wingless-type MMTV integration site family, member 16 (WNT16) or Wingless-type MMTV integration site family, member 5A (WNT5A) may be a polynucleotide such as a cDNA sequence or an mRNA sequence of WNT16 or WNT5A, or may be the WNT16 or WNT5A polypeptides. Accordingly the “expression” of WNT16 or WNT5A may refer to the expression of Wingless-type MMTV integration site family, member 16 or Wingless-type MMTV integration site family, member 5A cDNA, mRNA and/or polypeptide.

In certain embodiments the diagnostic method comprises comparing the levels of expression of at least two molecules associated with the Wnt signalling pathway with control levels of expression of these molecules. In certain embodiments the diagnostic method comprises comparing the levels of expression of at least three molecules associated with the Wnt signalling pathway with control levels of expression of these molecules.

In certain embodiments the marker molecules are selected from Secreted Frizzled-related Protein-1, paired-like homeodomain transcription factor 2 or lymphoid enhancer-binding factor 1. In certain embodiments the marker molecules are selected from Wingless-type MMTV integration site family, member 16 or Wingless-type MMTV integration site family, member 5A. It is anticipated that other molecules which are involved with or components of the Wnt signalling pathway may also be upregulated or down regulated in keratoconus, pellucid marginal degeneration or corneal ectasia following refractive surgery. The identification of other mRNA markers involved with or components of the Wnt receptor signalling pathway may be made using commercially-available PCR arrays which are able to compare the levels of expression of numerous genes associated with Wnt signalling and/or apoptosis. Additional markers molecules which may provide diagnostic include the soluble Wnt antagonists SFRP-2 and SFRP5.

The level of marker molecules which are involved with or are components of the Wnt signalling pathway, such as Secreted Frizzled-related Protein-1, paired-like homeodomain transcription factor 2 and/or lymphoid enhancer-binding factor 1, may be determined by detecting mRNA expression in cells or tissues of the subject using any quantitative or semi-quantitative methods available in the art, such as Northern blotting, dot blotting, real-time polymerase chain reaction, serial analysis of gene expression (SAGE) (Velculescu V E, Zhang L, Vogelstein B, Kinzler K W. (1995). “Serial analysis of gene expression”. Science 270 (5235): 484-7) and its variants such as SuperSAGE (Hideo Matsumura, Stefanie Reich, Akiko Ito, Hiromasa Saitoh, Sophien Kamoun, Peter Winter, Günter Kahl, Monika Reuter, Detlev H. Krüger, and Ryohei Terauchi (2003): “Gene expression analysis of plant host-pathogen interactions by SuperSAGE.” PNAS 100:15718-15723).

For example, real-time polymerase chain reaction is able to amplify, detect and quantify specific mRNA expression from a biological sample. The term polymerase chain reaction or PCR is intended to broadly encompass the varied techniques which utilise a primer or primers to selectively direct the action of a DNA polymerase on a template sequence to repeatedly produce (or amplify) specific DNA sequences. PCR commonly utilises pairs of primers which bind both strands of the DNA duplex, as this allows for the exponential amplification of the selected DNA strands; however it will be understood that asymmetric or single strand PCR may also be carried out using primers to only one DNA strand, although such techniques only allow for the linear amplification of the desired DNA sequence. Reverse transcription PCR may also be used in the context of the present method to identify SFRP1, PITX2, LEF1, WNT16 or WNT5A or other diagnostic mRNA sequences, convert them to cDNA and then allow for PCR amplification. Real time PCR, in which the process of amplification may be monitored and quantified using DNA binding dyes or labelled specific nucleotide sequence probes is also contemplated. Quantification of the levels of mRNA present in an initial sample are possible using RT-PCR techniques by first determining relative amounts of the relevant polynucleotide present in the exponential phase of reaction by plotting fluorescence against cycle number. For most accurate assessment, measurements of the relevant polynucleotide will be normalized against a housekeeping gene which is expressed at constant levels. The amounts of relevant polynucleotide in the initial sample is then determined by reference to a standard curve produced by. RT-PCR of serial dilutions of a known polynucleotide.

In general, the minimum quantity of RNA in a sample which is required for the detection of specific mRNA species using currently available detection techniques is of the order of 1 μg of total RNA. The quantity of total RNA extracted from discrete corneal scrapings is of the order of 2-4 μg of total RNA, allowing for a possible plurality of tests to be carried out from a single sample.

Commercially-available arrays for the PCR-based quantification of mRNA associated with the Wnt-signalling pathway in humans are provided by SABiosciences (formerly SuperArray Bioscience Corporation), for example Catalog Number PAHS-043.

Specific primers and probes for the amplification of marker molecule mRNAs are also available. For example, primer sequences for amplifying human SFRP1 polynucleotide sequences are described in Caldwell G M, et al. The Wnt antagonist sFRP1 in colorectal tumorigenesis, Cancer Res. (2004) 64(3):883-8.

Although specific primers are described herein to illustrate reagents which may be used to amplify SFRP1, PITX2 or LEF1 or other diagnostic RNA sequences, it will be recognised that any primers suitable for amplifying specific sequences of polynucleotides associated with Wnt signalling pathway for detection may be employed and are contemplated in the present method.

Systems suitable for carrying out real time polymerase chain reactions are readily commercially available, for example from Applied Biosystems (7500 Real Time PCR System) or Bio-Rad (iCycler).

Probes for the detection of amplified Wnt signalling pathway polynucleotide sequences are also commercially available, for example in kit form accompanied by relevant PCR primers and reporter dyes, such as the RT2 Profiler™ PCR Array (SABiosciences, formerly SuperArray Bioscience Corporation).

In addition to, or as an alternative to, the detection of any one or more polynucleotides associated with the Wnt signalling pathway, in certain embodiments the detection involves the detection of any one or more of SFRP1, PITX2, LEF1, WNT16 and/or WNT5A polypeptides. The level of certain molecules which are involved with or components of the Wnt signalling pathway, such as Secreted Frizzled-related Protein-1, paired-like homeodomain transcription factor 2 and/or lymphoid enhancer-binding factor 1, readily may be determined by detecting polypeptide expression in or on cells, in tissues or in extracellular secretions of the subject using any quantitative or semi-quantitative methods available in the art, including techniques which rely on antibodies for specific detection of polypeptides, such as Western blotting using an antibody which may be conjugated to a fluorophore or to a reporter enzyme, immunohistochemistry, or enzyme-linked immunosorbent assays, or by using techniques which rely on polypeptide sequence analysis such as semi-quantitative mass spectrometry techniques.

Where the polypeptide is a secreted polypeptide, levels of polypeptide may be detectable on the plasma membrane of and/or within corneal epithelial cells synthesising the polypeptide, as well as in the extracellular matrix surrounding these cells or in tears.

Commercially available antibodies which may be used for detecting and quantifying SFRP1 are available, for example the rabbit polyclonal to SFRP1 which is available from Abcam as catalogue number ab6673.

In certain embodiments, the methods provided involve a comparison of the level of expression of one or more Wnt signalling pathway associated marker molecules by corneal cells, such as corneal epithelial cells, of a subject with a control level of expression, wherein an elevated level of the one or more Wnt signalling pathway associated marker molecules by corneal cells of the subject compared to a control level indicates the subject has or is at risk of keratoconus, pellucid marginal degeneration or corneal ectasia following refractive surgery. Without wishing to be bound by any proposed mechanism of action, the experimental data presented in Table 2 suggests that individual subjects with the greatest rate of progression of keratoconus exhibited the highest elevation of expression of SFRP1, suggesting a correlation between the level of expression of SFRP1 and the rate of keratoconus progression in the subject.

In certain embodiments the method of diagnosis comprises the step of binding the marker molecule to a marker molecule detecting agent to form a complex, and detecting the level of the complex. For example, in embodiments in which the marker molecule is a polypeptide there may be a step of binding the marker molecule to a detecting agent, such as an antibody specific to the marker molecule, to form an antibody-marker molecule complex, the complex then being detected in the method. In another example, in embodiments in which the marker molecule is a polynucleotide there may be a step of hybridizing the marker molecule to a specific probe or primer to form a marker molecule polynucleotide/probe or primer hybrid which leads to the amplification and/or detection of the polynucleotide marker molecule.

Therapeutic Methods

The experimental results presented herein identify several marker molecules which are associated with the Wnt signalling pathway and the increased expression of which correlates with the presence of keratoconus in the subject. In particular, all subjects with keratoconus exhibited a considerable upregulation in corneal epithelial cells of the Wnt signalling pathway antagonist SFRP1, suggesting a reduction of Wnt signalling in the corneas of keratoconic subjects.

The recognition that there may be modulation, such as a reduction, of Wnt signalling in the corneas of keratoconic subjects, and that subjects with more rapidly progressing keratoconus may exhibit a greater level of Wnt signalling reduction leads to the insight that molecules which are Wnt signalling agonists may be used to counter a reduction in Wnt signalling in subjects with keratoconus, and may thereby modulate the progression of keratoconus and similar conditions in the subject.

Accordingly, in one aspect there is provided a method of treating or preventing keratoconus, pellucid marginal degeneration or corneal ectasia following refractive surgery in a subject, comprising administering to the subject an agonist of the Wnt signalling pathway.

The present inventors have demonstrated that elevated levels of SFRP1, PITX2 and LEF1 are associated with keratoconus, and that subjects with more rapidly progressing keratoconus exhibit a greater elevation of SFRP1 than subjects with a less rapidly progressing form of keratoconus. As SFRP1 is an antagonist of the Wnt signalling pathway, and the inhibition of the. Wnt signalling pathway is associated in other tissues with the induction of apoptosis, counteracting the observed elevation of SFRP1 and/or the reduction in Wnt signalling associated with elevated levels of SFRP-1 may provide a therapeutic or prophylactic benefit to subjects diagnosed with or at risk of developing keratoconus.

In certain embodiments the agonist of the Wnt signalling pathway reduces the levels of SFRP1 or antagonises the activity of SFRP1.

In certain embodiments the agent is an antibody, such as a neutralising antibody to SFRP1. The use of a neutralising antibody specific to SFRP1 to modulate SFRP1-mediated periodontitis and to hasten wound closure in palatal wounds in mice has been described previously, and accordingly the therapeutic modulation of SFRP1 polypeptide function by the administration of an SFRP1-specific antibody to the anterior cornea or corneal surface is anticipated to modulate SFRP1-mediated cellular processes in the cornea.

The term “antibody” is used in the broadest sense and specifically covers single monoclonal antibodies (including antagonist, and neutralizing antibodies) and antibody compositions with polyepitopic specificity as well as other recombinant molecules derived from these antibodies. The term “monoclonal antibody” as used herein refers to an antibody obtained from a population of substantially homogeneous antibodies, i.e., the individual antibodies comprising the population are identical except for possible naturally-occurring mutations that may be present in minor amounts. An “antibody” is also intended to encompass an antibody fragment which retains the ability to bind the relevant epitope. In certain embodiments the antibody may be of human origin, or may be a humanised antibody.

In other embodiments the antibody is an agonist of Wnt signalling, for example a Frizzled receptor agonist.

In certain embodiments the agonist of Wnt signalling is an antisense or RNAi which inhibits the expression of SFRP1 in the cornea. The use of antisense molecules delivered to the cornea to downregulate the corneal expression of particular polypeptides for therapeutic or tissue engineering purposes has been described in International patent application publication No WO 2005/053600 (Coda Therapeutics (NZ) Ltd), the entire contents of which is incorporated herein by reference. The complete sequence of human SFRP1 is known, for example HGNC: 10776; Entrez Gene: 6422; UniProt: Q8N474; or Ensembl: ENSG00000104332. Methods for generating antisense molecules or RNAi which are able to down regulate the expression of a polypeptide are well known to the skilled addressee, and once in possession of the sequence of SFRP1 the generation of antisense or RNAi is routine. For example, the RNAi knockdown of SFRP1 polypeptide expression in rats has been demonstrated in Wang et al., “Secreted Frizzled-related Protein 1 (SFRP1) Modulates Glucocorticoid Attenuation of Osteogenic Activities and Bone Mass”, Endocrinology (2005) as doi:10.1210/en.2004-1050.

Similarly, antisense or RNAi knockdown of other antagonists of Wnt signalling may provide an enhancement to the level of Wnt signalling in the cornea.

Antibodies may be administered to the cornea by topical application, for example in the form of aqueous drops, creams or in bandage or depot contact lenses, optionally in admixture with agents which increase the viscosity of the administered solution to increase residence time in the tears. Antibodies may also be administered to the cornea via subconjunctival injection.

Antisense or RNAi may be delivered to the cornea topically using established techniques for polynucleotide administration, or may be delivered as a bolus within the corneal stroma or subconjunctivally. An example of the successful knockdown of gene expression in the cornea is demonstrated in the successful knockdown of connexin expression in the cornea following subconjunctival delivery of an antisense molecule to connexin in WO 2005/053600 (supra).

In certain embodiments the agonist of the Wnt signalling pathway is an agonist of a marker molecule which has an increased expression in keratoconus. In other embodiments the agonist of the Wnt signalling pathway acts upstream or downstream in the Wnt signalling pathway of the molecule which is present at elevated levels. For instance, the activity of SFRP1 inhibiting Wnt may result in the activation of Glycogen synthase kinase-3 (GSK-3) phosphorylation of β-catenin, which in turn leads to an increased ubiquitination and proteolysis of β-catenin, thus reducing the amount of β-catenin mediated signalling in the Wnt signalling pathway. The use of a known GSK-3α and 3β inhibitor would thus antagonise the effects of SFRP1.

U.S. Pat. No. 7,105,532 entitled “Pyrazolo[3,4-C]Pyridines as GSK-3 Inhibitors” (Rawlings and Witherington, filed 19 Dec. 2001, the entire contents of which is incorporated by reference), discloses the synthesis of an inhibitor of glycogen synthase kinase-3 of the following formula:

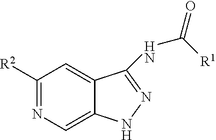

or a derivative thereof, wherein

R¹ is unsubstituted or substituted alkyl, unsubstituted or substituted cycloalkyl, unsubstituted or substituted alkenyl, unsubstituted or substituted cycloalkenyl, unsubstituted or substituted aryl, aralkyl wherein the aryl and the alkyl moieties may each independently be unsubstituted or substituted, aralkenyl wherein the aryl and alkenyl moieties may each independently be unsubstituted or substituted, unsubttituted or substituted heterocyclyl, or heterocyclylalkyl wherein the heterocyclyl and the alkyl moieties may each independently be unsubstituted or substituted; and

R² is unsubstituted or substituted aryl or unsubstituted or substituted heteroaryl.

Also disclosed is a compound of formula (I) or a derivative thereof wherein R¹ is unsubstituted or substituted alkyl, unsubstituted or substituted cycloalkyl, unsubstituted or substituted aryl, unsubstituted or substituted heterocyclyl, or heterocyclylalkyl wherein the heterocyclyl and the alkyl moieties may each independently be unsubstituted or substituted; and wherein R² is unsubstituted or substituted aryl or unsubstituted or substituted heteroaryl.

When R¹ is unsubstituted or substituted alkyl, examples include C₁₋₆alkyl, for example methyl, ethyl, propyl, butyl and iso-propyl.

When R¹ is unsubstituted or substituted cycloalkyl, examples include C₃₋₈cycloalkyl, for example cyclopropyl and cyclopentyl.

When R¹ is unsubstituted or substituted alkenyl, examples include C₂₋₆alkenyl.

When R¹ is unsubstituted or substituted aryl, examples include phenyl.

When R¹ is unsubstituted or substituted aralkyl, examples include benzyl and phenethyl.

When R¹ is unsubstituted or substituted aralkenyl, examples include styryl.

When R¹ is unsubstituted or substituted heterocyclyl, examples include fluryl, pyridyl and piperidinyl. A typical example of R¹ is piperidinyl.

When R¹ is unsubstituted or substituted heterocyclylalkyl, examples include piperidinylpropyl, piperazinylpropyl, morpholinylpropyl, pyrrolidinylpropyl and pyridylethyl. Suitably, R¹ is piperidinylpropyl, piperazinylpropyl and pyrrolidinylpropyl.

When R¹ is substituted alkyl, example substituents include halo, C₁₋₆alkoxy, carboxy, di(C₁₋₆alkyl)amino and phenoxy.

When R¹ is substituted aryl, example substituents include up to five groups independently selected from the list consisting of hydroxy, C₁₋₆alkoxy, di(C₁₋₆alkyl)amino, cyano, C₁₋₆alkyl, carboxy, C₁₋₆alkoxycarbonyl, C₁₋₆alkylaminocarbonyl, C₁₋₆alkylcarbonylamino, amino, halo, nitro and a substituent —R³NR⁴R⁵ wherein R³ is C₁₋₆alkylene and R⁴ and R⁵ are C₁₋₆alkyl, or R³ is C₁₋₆alkylene and R⁴ and R⁵ together with the nitrogen atom to which they are attached form a heterocyclic ring.

When R¹ is substituted heterocyclyl, example substituents include up to five groups independently selected from the list consisting of hydroxy, C₁₋alkoxy, di(C₁₋₆alkyl)amino, cyano, C₁₋₆alkyl, carboxy, C₁₋₆alkoxycarbonyl, C₁₋₆alkylaminocarbonyl, C₁₋₆alkylcarbonylamino, amino, halo, nitro and a substituent —R³NR⁴R⁵ wherein R³ is C₁₋₆ alkylene and R⁴ and R⁵ are C₁₋₆alkyl, or R³ is C₁₋₆ alkylene and R⁴ and R⁵ together with the nitrogen atom to which they are attached form a heterocyclic ring.

In some examples, R¹ is n-propyl, iso-propyl, cyclopropyl, cyclopentyl, 3-dimethylaminopropyl, 4-dimethylaminophenyl, 3-(pyrrolidin-1-yl)propyl, 3-(piperidin-1-yl)propyl, 3-(4-ethylpiperazin-1-yl)propyl, 1-methylpiperidin-4-yl and 4-[(pyrrolidin-1-yl)methyl]phenyl.

When R² is unsubstituted or substituted aryl, examples include phenyl.

When R² is unsubstituted or substituted heteroaryl, examples include pyridinyl, thienyl, furyl, quinolinyl and indolyl. In some examples, R² is pyridin-3-yl or quinolin-3-yl.

When R² is substituted aryl, example substituents include up to five groups independently selected from the list consisting of benzyloxy, halo, Calkyl, C₁₋₆alkoxy, C₁₋₃alkylenedioxy, C₁₋₆alkylcarbonylamino, perhaloC₁₋₆alkyl, nitro and perhaloC₁₋₆alkoxy.

When R² is substituted heteroaryl, example substituents include up to five groups independently selected from the list consisting of C₁₋₆alkoxy, halo, aryl and C₁₋₆alkyl. Suitably, R² is phenyl, 2-chlorophenyl, 2,3-difluorophenyl, 4-fluorophenyl, 2,3,4-trifluorophenyl, pyridin-3-yl, 5-phenylpyridin-3-yl, 6-methylpyridin-3-yl, 6-methyoxypyridin-3-yl and quinolin-3-yl.

This molecule in the form of N-(5-phenyl-1H-pyrazolo[3,4-C]pyridazino-3 yl)-4-morpholine butanamide and with activity as a GSK-3α and 3β inhibitor was administered topically to the eye to antagonise the effects of SFRP1 in raising intraocular pressure in an animal model of glaucoma (Wang et al. (2008) J Clin Invest 118:1056-1064).

Accordingly, this compound and other GSK-3 inhibitors of formula (I) as described in U.S. Pat. No. 7,105,532 may be used as an agonist of the Wnt signalling pathway in the cornea.

In the context of this specification, the term “comprising” means “including, but not necessarily solely including”. Furthermore, variations of the word “comprising” such as “comprise” and “comprises” have correspondingly varied meanings.

Throughout this specification, reference to “a” or “one” element does not exclude the plural, unless context determines otherwise. For instance, reference to “a subject” should not be read as excluding the possibility of multiple subjects.

### EXAMPLES

### Example 1

### Quantification of the change of Gene Expression in the Corneal Epithelium of Keratoconus Subjects

Samples of corneal epithelium were collected from KC patients undergoing corneal transplantation and 6 age-matched controls undergoing photorefractive keratectomy (PRK). The clinical characteristics of the KC patients including age, best spectacle corrected visual acuity (BSCVA), mean keratometry and rate of corneal steepening were documented at the time of surgery and are set out in Table 2.

All keratoconus subjects exhibited poor vision which was not correctable by contact lenses. The rate of progression of keratoconus in the subjects was assessed by quantifying the amount of corneal steepening over time between assessments.

Tetracaine 1% (Minims) and Chloramphenicol 0.5% (Minims) eye drops were administered every ten minutes for 30 minutes prior to surgery. An 8 mm trephine was used to mark the epithelial surface and an 8 mm diameter of central epithelium was removed with a hockey blade and placed immediately into Trizol (InVitrogen) and stored at −80° C. until processed. No alcohol was used on the epithelial surface in either group. All procedures were done with consent and following approval from the South East & Illawarra Area Health Service Human Ethics Committee.

Total RNA was prepared using an initial Trizol extraction followed by purification with an RNAqueous-Micro Kit (Ambion) according to the manufacturers instructions. After extraction; the quality of the RNA was assessed by electrophoresis on 1.5% RNAse-free agarose gels (Research Organics) and visualised with ethidium bromide. Samples were then stored at −80° C.

Quantitative RT-PCR was performed using manufacturers protocols for the RT² Profiler™ PCR Array (Human WNT Signaling Pathway) (PAHS-043, Superarray, Gaithersburg, Md., USA). This array allowed the determination of the expression profile of 84 genes associated with Wnt-mediated signal transduction. A list of the genes tested for in this assay, together with their Unigene and GeneBank database accession numbers is provided in Table 4.

Relative gene expressions were calculated using the 2^{-ΔΔCt} method, in which Ct indicates the fractional cycle number where the fluorescent signal reaches detection threshold. The ‘delta-delta’ method (Pfaffl, 2001) uses the normalized ΔCt value of each sample, calculated using a total of five endogenous control genes (B2M, HPRT1, RPL13A, GAPDH, and ACTS). Fold change values were then presented as average fold change=2 (average^{ΔΔct}) for genes in KC relative to control samples. Detectable PCR products were obtained for 72/84 genes for all subjects (defined as requiring <35 cycles). The genes not detected were Dickkopf Homolog-1 (Xenopus laevis) (DKK1), Fibroblast Growth Factor-4 (FGF4), Follicle stimulating hormone beta polypeptide (FSHB), Frizzled Homolog 2 (Drosophila) (FZD2), Pygopus Homolog 1 (PYG01), secreted Frizzled Related Protein-4 (SFRP4), SRY (Sex determining region Y)-box 17 (SOX17), Wnt Inhibitory Factor-1 (WIF1), Wingless-type MMTV integration site family, member 1 (WNT1), Wingless-type MMTV integration site family, member 2 (WNT2), Wingless-type MMTV integration site family, member 8A (WNT8A), and Wingless-type MMTV integration site family, member 11 (WNT11). The statistical significance of differences in gene expression between KC and control groups was calculated using a two-tailed student's t-test (p<0.05). Genes not detected were presumably not expressed at detectable levels using this technology.

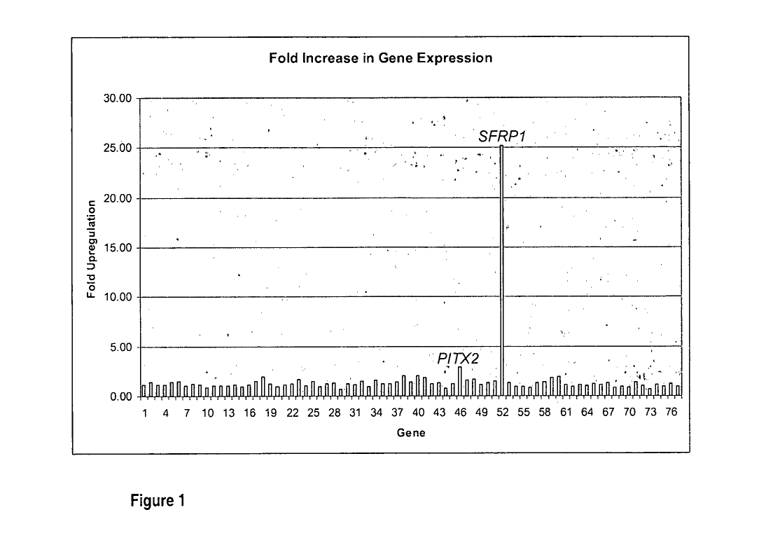

Using RT-PCR microarrays, Lymphoid enhancer-binding factor-1 (LEF1), Paired-like homeodomain transcription factor 2 (PITX2) and Secreted Frizzled related protein-1 (SFRP1) were up-regulated more than 2-fold in KC compared to control epithelium (Table 1 and FIG. 1). SFRP1 was strongly upregulated in all KC patients compared to controls (P=0.019) (range 9.12-fold to 98.6-fold; p=0.019) (Table 1 and FIG. 1). Expression of the five housekeeping genes (82M, HPRT1, RPL 13A, GAPDH and ACTB) was not significantly different between KC and control patients (Table 1).

Subsequent experiments with a total of 13 keratoconus subjects and 5 control subjects demonstrated that SFRP-1 was upregulated in all cases of keratoconus between 3 and 75 fold (and on average 13 fold) compared to the average level of expression in controls. LEF-1 was upregulated at least 2 fold in keratoconus subjects, and PITX2 and SFRP-2 were upregulated slightly less than 2 fold. In addition, keratoconic subjects were found to have downregulation of between 2 to 2.5 fold expression of the marker molecules WNT16 and WNT5A when compared to the average level of expression of these markers in control subjects. Repeat testing of the same samples generated consistent results.

TABLE 1
Up-regulated genes (fold change >2.00) between keratoconus &
control corneal epithelium

| | Gene | | |
|---|---|---|---|
| Gene Name | Symbol | ^{a}Fold Change | ^{b}P-value |
| Up-regulated genes | | | |
| Lymphoid enhancer- | LEF1 | 2.08 | NS (P = 0.14) |
| binding factor 1 | | | |
| Paired-like homeodomain | PITX2 | 2.89 | NS (P = 0.09) |
| transcription factor 2 | | | |
| Secreted frizzled-related | SFRP1 | 25.25 | P = 0.019 |
| protein 1 | | | |
| Housekeeping control genes | B2M | 0.75 | NS (P = 0.24) |
| | HPRT1 | 1.13 | NS (P = 0.39) |
| | RPL13A | 1 | NS (P = 0.15) |
| | GAPDH | 1.25 | NS (P = 0.75) |
| | ACTB | 0.94 | NS (P = 0.35) |

^{a}Average Fold Change: 2(average^{ΔΔCt}) for genes in KC relative to control samples.
^{b}P-value: 2-tailed t-test, p < 0.05; NS—not significant

All KC patients had advanced keratoconus, with keratometry greater than 65D (Table 2). There was evidence of recent corneal steepening of >1.5D (using keratometry) in Cases 1, 2 and 4.

In the patients less than 30 years of age, SFRP1 fold increases of 37.97 and 98.6 were found (Table 2). SFRP1 expression was associated with age and possibly the rate of progression of the KC, although statistical comparison of SFRP1 fold changes and patient characteristics was not possible due to the small number of samples.

TABLE 2
Clinical features of keratoconic patients

| | Patient # | | | |
|---|---|---|---|---|
| | 1 | 2 | 3 | 4 |
| Age | 28 | 24 | 47 | 34 |
| Mean keratometry | >65D | Not obtained | >60D | 63D |
| ^{a}BSCVA | CF | 6/60 | 6/60 | 6/36 |
| Δ in mean keratometry | >2D | 3D | Not obtained | 1.5D |
| 1 years | | | | |
| ^{b}Fold Change SFRP1 | 98.6 | 37.95 | 11.91 | 9.12 |

^{a}BSCVA: best spectacle corrected visual acuity; CF—count fingers
^{b}Fold Change SFRP1: from quantitative RT-PCR microarray data Table 1

TABLE 3
Up-regulated genes in individual keratoconus epithelium samples

| | | | | | Pooled |
|---|---|---|---|---|---|
| Gene | Patient 1 | Patient 2 | Patient 3 | Patient 4 | KC |
| CXXC4 | 2.14 | 1.46 | 1.98 | 2.55 | 1.99 |
| JUN kinase | 1.68 | 2.77 | 2.61 | 1.32 | 2.0 |
| LEF1 | 1.25 | 1.56 | 2.35 | 3.98 | 2.07 |
| LRP5 | 2.47 | 1.26 | 1.62 | 2.27 | 1.84 |
| PITX2 | −1.98 | 13.85 | 1.93 | 5.19 | 2.89 |
| SFRP1 | 98.6 | 37.95 | 11.91 | 9.12 | 25.25 |
| TLE2 | 2.32 | 1.2 | 2.57 | 1.57 | 1.83 |
| WISP1 | 1.63 | 2.46 | 1.94 | 1.95 | 1.97 |

Numerical values represent Fold Change in expression from quantitative RT-PCR microarray data.

To further characterise the expression of SFRP1 in KC and normal cornea, specimens from KC patients undergoing corneal transplantation (n=3) and control corneas (n=3) were examined using immunohistochemistry. Briefly, paraffin sections of cornea were dewaxed in xylenes, and hydrated through a graded series of alcohols. Following antigen retrieval in 0.01M citrate buffer (pH 6.0) for 10 mins, sections were rinsed in 0.1M phosphate buffered saline (pH7.4), then incubated in 10% normal goat serum, followed by overnight incubation at 4° C. in mouse anti-human SFRP1 antibody (4 ug/ml, Santa-Cruz). Immunolabelling was visualized using Alex488 conjugated goat anti-mouse antibody (1:1000, Molecular Probes), nuclei were stained with Hoechst 33342 or propidium iodide and sections were examined using a Zeiss LCM5 confocal microscope.

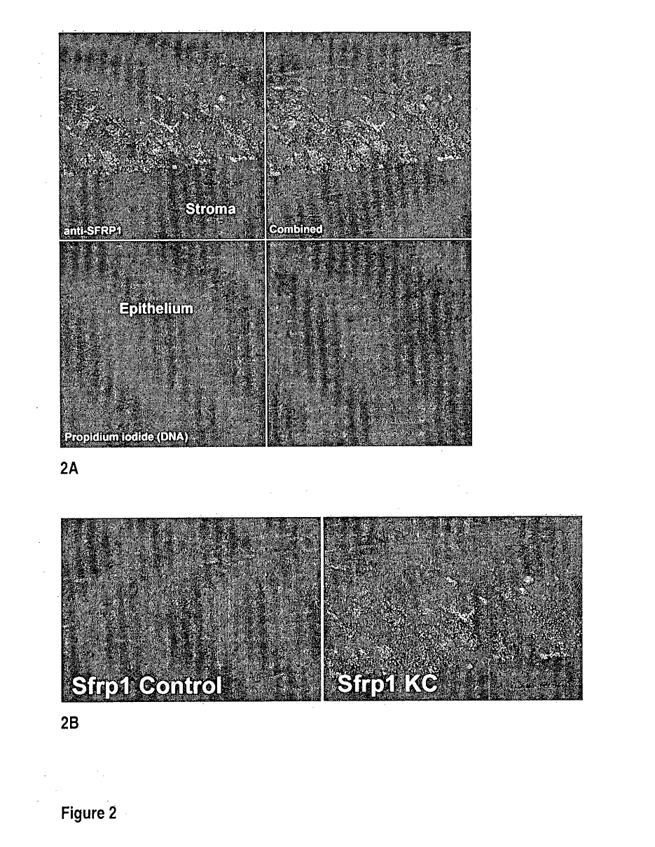

The immunolocalization of SFRP1 protein in KC compared to normal corneal epithelium is shown in FIG. 2. SFRP1 was seen mostly in the basal epithelium, although in some areas, more superficial corneal epithelium also expressed SFRP1. Minimal SFRP1 immunostaining was observed in control corneas, and was similar to the non-specific Ig control.

TABLE 4

| Position | UniGene | GenBank | Symbol | Description |
|---|---|---|---|---|
| A01 | Hs.515053 | NM_001130 | AES | Amino-terminal enhancer of split |
| A02 | Hs.158932 | NM_000038 | APC | Adenomatosis polyposis coli |
| A03 | Hs.592082 | NM_003502 | AXIN1 | Axin 1 |
| A04 | Hs.415209 | NM_004326 | BCL9 | B-cell CLL/lymphoma 9 |
| A05 | Hs.643802 | NM_033637 | BTRC | Beta-transducin repeat containing |
| A06 | Hs.17631 | NM_003468 | FZD5 | Frizzled homolog 5 (Drosophila) |
| A07 | Hs.523852 | NM_053056 | CCND1 | Cyclin D1 |
| A08 | Hs.376071 | NM_001759 | CCND2 | Cyclin D2 |
| A09 | Hs.534307 | NM_001760 | CCND3 | Cyclin D3 |
| A10 | Hs.529862 | NM_001892 | CSNK1A1 | Casein kinase 1, alpha 1 |
| A11 | Hs.631725 | NM_001893 | CSNK1D | Casein kinase 1, delta |
| A12 | Hs.646508 | NM_022048 | CSNK1G1 | Casein kinase 1, gamma 1 |
| B01 | Hs.446484 | NM_001895 | CSNK2A1 | Casein kinase 2, alpha 1 polypeptide |
| 602 | Hs.208597 | NM_001328 | CTBP1 | C-terminal binding protein 1 |
| B03 | Hs.501345 | NM_022802 | CTBP2 | C-terminal binding protein 2 |
| B04 | Hs.476018 | NM_001904 | CTNNB1 | Catenin (cadherin-associated protein), |
| | | | | beta 1, 88 kDa |
| B05 | Hs.463759 | NM_020248 | CTNNBIP1 | Catenin, beta interacting protein 1 |
| B06 | Hs.12248 | NM_025212 | CXXC4 | CXXC finger 4 |
| B07 | Hs.19156 | NM_014992 | DAAM1 | Dishevelled associated activator of |
| | | | | morphogenesis 1 |
| B08 | Hs.446249 | NM_033425 | DIXDC1 | DIX domain containing 1 |
| B09 | Hs.40499 | NM_012242 | DKK1 | Dickkopf homolog 1 (Xenopus laevis) |
| B10 | Hs.74375 | NM_004421 | DVL1 | Dishevelled, dsh homolog 1 (Drosophila) |
| B11 | Hs.118640 | NM_004422 | DVL2 | Dishevelled, dsh homolog 2 (Drosophila) |
| B12 | Hs.517517 | NM_001429 | EP300 | E1A binding protein p300 |
| C01 | Hs.484138 | NM_012300 | FBXW11 | F-box and WD repeat domain containing |
| | | | | 11 |
| C02 | Hs.494985 | NM_012164 | FBXW2 | F-box and WD repeat domain containing 2 |
| C03 | Hs.1755 | NM_002007 | FGF4 | Fibroblast growth factor 4 (heparin |
| | | | | secretory transforming protein 1, Kaposi |
| | | | | sarcoma oncogene) |
| C04 | Hs.283565 | NM_005438 | FOSL1 | FOS-like antigen 1 |
| C05 | Hs.58611 | NM_003593 | FOXN1 | Forkhead box N1 |
| C06 | Hs.126057 | NM_005479 | FRAT1 | Frequently rearranged in advanced T-cell |
| | | | | lymphomas |
| C07 | Hs.128453 | NM_001463 | FRZB | Frizzled-related protein |
| C08 | Hs.36975 | NM_000510 | FSHB | Follicle stimulating hormone, beta |
| | | | | polypeptide |
| C09 | Hs.94234 | NM_003505 | FZD1 | Frizzled homolog 1 (Drosophila) |
| C10 | Hs.142912 | NM_001466 | FZD2 | Frizzled homolog 2 (Drosophila) |
| C11 | Hs.40735 | NM_017412 | FZD3 | Frizzled homolog 3 (Drosophila) |
| C12 | Hs.19545 | NM_012193 | FZD4 | Frizzled homolog 4 (Drosophila) |
| D01 | Hs.292464 | NM_003506 | FZD6 | Frizzled homolog 6 (Drosophila) |
| D02 | Hs.173859 | NM_003507 | FZD7 | Frizzled homolog 7 (Drosophila) |
| D03 | Hs.302634 | NM_031866 | FZD8 | Frizzled homolog 8 (Drosophila) |
| D04 | Hs.466828 | NM_019884 | GSK3A | Glycogen synthase kinase 3 alpha |
| D05 | Hs.445733 | NM_002093 | GSK3B | Glycogen synthase kinase 3 beta |
| D06 | Hs.525704 | NM_002228 | JUN | Jun oncogene |
| D07 | Hs.229335 | NM_001039570 | KREMEN1 | Kringle containing transmembrane protein 1 |
| D08 | Hs.555947 | NM_016269 | LEF1 | Lymphoid enhancer-binding factor 1 |
| D09 | Hs.6347 | NM_002335 | LRP5 | Low density lipoprotein receptor-related |
| | | | | protein 5 |
| D10 | Hs.584775 | NM_002336 | LRP6 | Low density lipoprotein receptor-related |
| | | | | protein 6 |
| D11 | Hs.202453 | NM_002467 | MYC | V-myc myelocytomatosis viral oncogene |
| | | | | homolog (avian) |
| D12 | Hs.592059 | NM_033119 | NKD1 | Naked cuticle homolog 1 (Drosophila) |
| E01 | Hs.208759 | NM_016231 | NLK | Nemo-like kinase |
| E02 | Hs.643588 | NM_000325 | PITX2 | Paired-like homeodomain transcription |
| | | | | factor 2 |
| E03 | Hs.386453 | NM_022825 | PORCN | Porcupine homolog (Drosophila) |
| E04 | Hs.483408 | NM_002715 | PPP2CA | Protein phosphatase 2 (formerly 2A), |
| | | | | catalytic subunit, alpha isoform |
| E05 | Hs.467192 | NM_014225 | PPP2R1A | Protein phosphatase 2 (formerly 2A), |
| | | | | regulatory subunit A, alpha isoform |
| E06 | Hs.256587 | NM_015617 | PYGO1 | Pygopus homolog 1 (Drosophila) |
| E07 | Hs.647774 | NM_021205 | RHOU | Ras homolog gene family, member U |
| E08 | Hs.401388 | NM_021627 | SENP2 | SUMO1/sentrin/SMT3 specific peptidase 2 |
| E09 | Hs.213424 | NM_003012 | SFRP1 | Secreted frizzled-related protein 1 |
| E10 | Hs.105700 | NM_003014 | SFRP4 | Secreted frizzled-related protein 4 |
| E11 | Hs.500822 | NM_022039 | FBXW4 | F-box and WD repeat domain containing 4 |
| E12 | Hs.396783 | NM_004252 | SLC9A3R1 | Solute carrier family 9 (sodium/hydrogen |
| | | | | exchanger), member 3 regulator 1 |
| F01 | Hs.98367 | NM_022454 | SOX17 | SRY (sex determining region Y)-box 17 |
| F02 | Hs.389457 | NM_003181 | T | T, brachyury homolog (mouse) |
| F03 | Hs.573153 | NM_003202 | TCF7 | Transcription factor 7 (T-cell specific, |
| | | | | HMG-box) |
| F04 | Hs.516297 | NM_031283 | TCF7L1 | Transcription factor 7-like 1 (T-cell |
| | | | | specific, HMG-box) |
| F05 | Hs.197320 | NM_005077 | TLE1 | Transducin-like enhancer of split 1 |
| | | | | (E(sp1) homolog, Drosophila) |
| F06 | Hs.332173 | NM_003260 | TLE2 | Transducin-like enhancer of split 2 |
| | | | | (E(sp1) homolog, Drosophila) |
| F07 | Hs.284122 | NM_007191 | WIF1 | WNT inhibitory factor 1 |
| F08 | Hs.492974 | NM_003882 | WISP1 | WNT1 inducible signaling pathway protein 1 |
| F09 | Hs.248164 | NM_005430 | WNT1 | Wingless-type MMTV integration site |
| | | | | family, member 1 |
| F10 | Hs.121540 | NM_025216 | WNT10A | Wingless-type MMTV integration site |
| | | | | family, member 10A |
| F11 | Hs.108219 | NM_004626 | WNT11 | Wingless-type MMTV integration site |
| | | | | family, member 11 |
| F12 | Hs.272375 | NM_057168 | WNT16 | Wingless-type MMTV integration site |
| | | | | family, member 16 |
| G01 | Hs.567356 | NM_003391 | WNT2 | Wingless-type MMTV integration site |
| | | | | family member 2 |
| G02 | Hs.258575 | NM_004185 | WNT2B | Wingless-type MMTV integration site |
| | | | | family, member 2B |
| G03 | Hs.445884 | NM_030753 | WNT3 | Wingless-type MMTV integration site |
| | | | | family, member 3 |
| G04 | Hs.336930 | NM_033131 | WNT3A | Wingless-type MMTV integration site |
| | | | | family, member 3A |
| G05 | Hs.591521 | NM_030761 | WNT4 | Wingless-type MMTV integration site |
| | | | | family, member 4 |
| G06 | Hs.652181 | NM_003392 | WNT5A | Wingless-type MMTV integration site |
| | | | | family, member 5A |
| G07 | Hs.306051 | NM_032642 | WNT5B | Wingless-type MMTV integration site |
| | | | | family, member 5B |
| G08 | Hs.29764 | NM_006522 | WNT6 | Wingless-type MMTV integration site |
| | | | | family, member 6 |
| G09 | Hs.72290 | NM_004625 | WNT7A | Wingless-type MMTV integration site |
| | | | | family, member 7A |
| G10 | Hs.512714 | NM_058238 | WNT7B | Wingless-type MMTV integration site |
| | | | | family, member 7B |
| G11 | Hs.591274 | NM_058244 | WNT8A | Wingless-type MMTV integration site |
| | | | | family, member 8A |
| G12 | Hs.149504 | NM_003395 | WNT9A | Wingless-type MMTV integration site |
| | | | | family, member 9A |
| H01 | Hs.534255 | NM_004048 | B2M | Beta-2-microglobulin |
| H02 | Hs.412707 | NM_000194 | HPRT1 | Hypoxanthine phosphoribosyltransferase |
| | | | | 1 (Lesch-Nyhan syndrome) |
| H03 | Hs.546356 | NM_012423 | RPL13A | Ribosomal protein L13a |
| H04 | Hs.544577 | NM_002046 | GAPDH | Glyceraldehyde-3-phosphate |
| | | | | dehydrogenase |
| H05 | Hs.520640 | NM_001101 | ACTB | Actin, beta |
| H06 | N/A | N/A | HGDC | Human Genomic DNA Contamination |
| H07 | N/A | N/A | RTC | Reverse Transcription Control |
| H08 | N/A | N/A | RTC | Reverse Transcription Control |
| H09 | N/A | N/A | RTC | Reverse Transcription Control |
| H10 | N/A | N/A | PPC | Positive PCR Control |
| H11 | N/A | N/A | PPC | Positive PCR Control |
| H12 | N/A | N/A | PPC | Positive PCR Control |

### Grouping of Markers in Array

Frizzled-1 Signaling Pathway: AXIN1, DIXDC1, DVL1, DVL2, FZD3, FZD4, FZD6, FZD7, FZD8.
Frizzled-2 Signaling Pathway: WNT1, WNT2, WNT2B, WNT3, WNT3A, WNT4, WNT5A, WNT5B, WNT6, WNT7A, WNT7B, WNT8A, WNT9A, WNT10A, WNT11, WNT16

### Regulation of Wnt Receptor Signaling Pathway:

Negative Regulation of Wnt Receptor Signaling Pathway. CXXC4, DKK1, FRZB (FRP-3), FSHB, NLK.

Wnt Binding Antagonists: SFRP1, SFRP4 (FRP-4).

Other Regulators of Wnt Signaling. PPP2CA, PPP2R1A, SENP2, TCF7L1.

Regulation of Growth and Proliferation:

Regulation of Growth: PPP2CA, PPP2R1A, WISP1.

Regulation of Cell Proliferation: CTBP1, CTBP2, LRP5.

Other Genes Related to Growth and Proliferation: FRAT1.

Regulation of the Cell Cycle: APC, CCND1, CCND2, CCND3, FGF4, PPP2CA, PPP2R1A.

### Regulation of Transcription:

Transcription Factors: EP300, FOSL1 (Fra-1), FOXN1, JUN, MYC, PITX2, T (Brachyury), TCF7L1.

Other Regulators of Transcription: AES (TLE/Groucho), CTNNBI (catenin ?), CTNNBIP1 (ICAT), LEF1, NLK, PPP2CA, PPP2R1A, PYG01, SOX17, TCF7 (Tcf-1), TLE1, TLE2, WNT1.

### Protein Modification Activities:

Protein Kinase Activity CSNK1A1, CSNK1 D, CSNK1G1, CSNK2A1, CTBP1, GSK3A (GSKalpha), GSK3B (GSKbeta), NLK, WIF1.

Protein Phosphatase Type 2A Complex. PPP2CA, PPP2R1A.

Protein Ubiquitination: BTRC (b-TrCP), FBXW11, FBXW2, FBXW4.

Other Genes Related to Wnt Signaling: BCL9, FZD5 (FZD5), DAAM1, FZD1, FZD2, KREMEN1, LRP6, NKD1, PORCN, RHOU, SLC9A3R1.

### Example 2

### Diagnosis of Keratoconus Using expression of SFRP1

Subjects for diagnosis may be selected on the basis of previous clinical diagnosis, for example by corneal topography, where the methods described herein may provide a confirmatory diagnosis and may identify whether the condition will progress rapidly or slowly. In addition, subjects intending to undergo refractive surgery, such as LASIK, may be screened. This diagnostic test may also be used for screening donor corneas prior to corneal transplantation procedures. This diagnostic test may also be carried out on subjects following refractive surgery in which there is suspected corneal ectasia.

A sample of full thickness epithelium of approximately 1 mm² area from one or both corneas is taken under local anaesthetic using a sterile blade or scraper. Corneal epithelial samples are taken from the region overlying any corneal topographical anomaly, or from the corneal periphery. A similarly sized sample of conjunctival epithelium may also be taken. The subject's eye is given prophylactic antibiotics and non-steroidal anti-inflammatory agents, and bandaged to allow healing of the epithelial wound.

The level of expression of one or more relevant marker molecules is determined using quantitative real time PCR techniques as described in Example 1. The relative level of expression of SFRP1 for example, is determined and compared with level of SFRP1 expression from control subjects.

In subjects in which the level of expression of one or more marker molecules is elevated at least 2-fold over control levels, or elevated to a statistically significant amount, a diagnosis of keratoconus, pellucid marginal degeneration or corneal ectasia may be made.

In subjects in which a diagnosis of keratoconus, pellucid marginal degeneration or corneal ectasia is made, and in particular in subjects exhibiting strong elevation of expression of one or more marker molecules and thus considered to be at risk of rapid progression, therapeutic methods to slow or prevent further corneal topography change may be commenced. These therapies may include riboflavin-mediated corneal collagen cross linking therapy, or the therapy set out in Example 3.

### Example 3

### Treatment of Keratoconus, of Pellucid Marginal Degeneration or of Corneal Ectasia Following refractive Surgery

Subjects are diagnosed with keratoconus, pellucid marginal degeneration or corneal ectasia following refractive surgery using the diagnostic methods described herein and/or using standard diagnostic criteria. For example, a keratoconic subject with loss of best spectacle corrected vision of at least 80% would be eligible for the treatment.

In certain methods, subjects are topically administered a sterile aqueous solution of an agent which comprises neutralising antibodies to SFRP1 polypeptide to the corneal surface. In other methods, the agent comprises antisense molecules to SFRP1 or RNAi to SFRP1 and is administered to the eye using the general methods described in WO 2005/053600.

This administration may be formulated as aqueous eye drops, gels or creams, or may be provided in a depot such as a hydrogel lens which substantially covers the corneal surface or a hydrogel depot which may be inserted between the cornea and the lower lid, which provide a sustained release of agent to the corneal surface. It may be necessary to provided repeated instillation of agent in order to maintain an appropriate concentration of agent at the corneal surface.

In other methods subjects are administered a GSK-3 inhibitor in the form of N-(5-phenyl-1H-pyrazolo[3,4-C]pyridazino-3 yl)-4-morpholine butanamide topically to the eye, as described in Wang et al. (2008) J Clin Invest 118:1056-1064 in the context of treating glaucoma.

The progress of therapy, as determined by slowing or halting of changes in corneal topography is monitored with serial corneal topography, refraction and pachymetry.

### Example 4

### Detection of SFRP-1 Polypeptide Expression in Tear Samples

Example 1 demonstrated that mRNA marker molecules associated with the Wnt signalling pathway could be identified and quantified in a corneal cell sample. This example demonstrates the identification of polypeptide marker molecules in a tear sample.

Tear samples were obtained from control subjects and subjects diagnosed as described in Example 1 with keratoconus to investigate whether the expression of polypeptide marker molecules could be detected from the tears.

Basal tear samples were collected from the temporal meniscus by glass microcapillary tube over a five minute period. Approximately 7 to 10 microlitres of tears in total was collected from each subject. Tear sample were frozen at −80 degrees Celsius immediately following collection and stored for no longer than 6 months before analysed. Larger tear samples could be obtained by repeated sampling over a single visit or multiple visits. Previous tear collection studies have identified that there were only negligible numbers of corneal cells present in the tear samples when collected using this technique.

“Flush tears” were also collected for testing, in which 10 microlitres of phosphate buffered saline was instilled into the tear film and then the combined phosphate buffered saline and basal tears collected and frozen as described above.

Five microlitres of individual tear samples were diluted with 5× concentrated loading buffer comprising 2-mercaptoethanol and heated to 95 degrees Celsius in a water bath for five minutes to denature polypeptides in the sample. The denatured samples were loaded onto a 12% SDS polyacrylamide gel and run with a molecular weight marker at 4 degrees Celsius at a constant voltage of 120 V for 2 hours. If desired for quantitative determinations; known amounts of recombinant SFRP-1 (AbCam) may be run at the same time to provide a standard curve within the gel.

The gel was then rinsed and polypeptides transferred to a polyvinylidene difluoride 0.4 micrometer membrane (INVITROLON™, Invitrogen) which had been prepared according to the manufacturer's instructions for 2 hours at 4 degrees Celsius at a constant current of 250 mA.

The membrane with transferred polypeptides was blocked in 5% bovine serum albumin in Tween/Tris buffered saline overnight at 4 degrees celsius, and rinsed extensively. The rinsed membrane was incubated with primary antibody to SFRP-1 (Santa Cruz Biotechnology, Inc. rabbit polyclonal antibody supplied at 200 microgram per ml and diluted 1:100) overnight at 4 degrees Celsius, and then rinsed extensively in Tween/Tris buffered saline. The membrane was then incubated with secondary antibody (Upstate Biotechnology, goat anti rabbit conjugated to horse, radish peroxidase, diluted 1:15000) for 2 hours at room temperature, and extensively rinsed in Tween/Tris buffered saline. Labelling as detected by peroxidase activity was visualised by chemiluminescence (Pierce Dura West ECL) and detected with GeneSnap imaging software.

SFRP-1 was detectable in samples from keratoconic and control subjects using this technique. Experiments to provide quantitative assessment of the level of expression of SFRP-1 are underway and the detection and quantitation of LEF-1, for example using a sheep anti-LEF-1 polyclonal antibody (LEF1 antibody (ab28316), AbCam) in tear samples are planned.

Alternative detection and quantitation techniques for marker molecules such as Mass spectrometry analysis may also utilise tear samples which have been separated by electrophoresis techniques.

1. A method of diagnosis of (i) corneal ectasia following refractive surgery, or (ii) keratoconus, or (iii) pellucid marginal degeneration in a subject, the method comprising comparing the level of expression of a marker molecule associated with the Wnt signalling pathway by corneal cells with a control level of expression of the molecule, wherein an elevated or lower level of expression of the molecule associated with the Wnt signalling pathway in the subject indicates the subject has or is at risk of developing corneal ectasia following refractive surgery, or keratoconus, or pellucid marginal degeneration.

2. The method according to claim 1, wherein the molecule associated with the Wnt signalling pathway is expressed at an elevated level over a control level and is selected from Secreted Frizzled-related Protein 1, Paired-like homeodomain transcription factor 2, Lymphoid enhancer-binding factor 1.

3. The method according to claim 1, wherein the molecule associated with the Wnt signalling pathway is expressed at a lower level to a control level and is selected from Wingless-type MMTV integration site family, member 16 or Wingless-type MMTV integration site family, member 5A.

4. The method according to claim 1, wherein the corneal cells are corneal epithelial cells, conjunctival epithelial cells or keratocytes.

5. The method according to claim 2, wherein the expression of Secreted Frizzled-related Protein 1, Paired-like homeodomain transcription factor 2 or Lymphoid enhancer-binding factor 1 is expression of Secreted Frizzled-related Protein 1, Paired-like homeodomain transcription factor 2 or Lymphoid enhancer-binding factor 1 mRNA.

6. The method according to claim 2, wherein the expression of Secreted Frizzled-related Protein 1, Paired-like homeodomain transcription factor 2 or Lymphoid enhancer-binding factor 1 is expression of Secreted Frizzled-related Protein 1, Paired-like homeodomain transcription factor 2 or Lymphoid enhancer-binding factor 1 polypeptide.

7. The method according to claim 3, wherein the expression of Wingless-type MMTV integration site family, member 16 or Wingless-type MMTV integration site family, member 5A is expression of Wingless-type MMTV integration site family, member 16 or Wingless-type MMTV integration site family, member 5A mRNA.

8. The method according to claim 3, wherein the expression of Wingless-type MMTV integration site family, member 16 or Wingless-type MMTV integration site family, member 5A is expression of Wingless-type MMTV integration site family, member 16 or Wingless-type MMTV integration site family, member 5A polypeptide.

9. The method according to claim 2, wherein the elevated level of expression is at least 2 fold level of expression over the control level.

10. The method according to claim 3, wherein the lower level of expression is at least 2 fold level of expression lower than the control level.

11. The method of claim 1, wherein the marker molecule is obtained from or present in a tear sample from the subject.

12. A method of treating (i) corneal ectasia following refractive surgery, or (ii) keratoconus, or (iii) pellucid marginal degeneration in a subject, comprising administering to the subject a modulator of the Wnt signalling pathway.

13. The method according to claim 12, wherein the modulator of the Wnt signalling pathway is an agonist of the Wnt signalling pathway.

14. The method according to claim 12, wherein the modulator of the Wnt signalling pathway is an antagonist of Secreted Frizzled-related Protein 1, Paired-like homeodomain transcription factor 2 or Lymphoid enhancer-binding factor 1.

15. The method according to claim 14, wherein the antagonist is an antibody, an antisense molecule or an RNAi:

16. The method according to claim 14, wherein the antagonist is a pyrazolo[3,4-C]pyridine or lithium.

17. The method according to claim 16, wherein the pyrazolo[3,4-C]pyridine is N-(5-phenyl-1H-pyrazolo[3,4-C]pyridazino-3 yl)-4morpholine butanamide.

18. The method according to claim 2, wherein the corneal cells are corneal epithelial cells, conjunctival epithelial cells or keratocytes.
